(19) United States
(12) Patent Application Publication
(10) Pub. No.: US 2024/0395382 A1
(43) Pub. Date: Nov. 28, 2024

(54) CAP FOR OPTICAL ILLUMINATION ON A CONTAINER

(71) Applicant: VCCB Holdings, Inc., Wilmington, DE (US)

(72) Inventors: Joshua Seth Wachman, Newton, MA (US); David Loring Rose, Brookline, MA (US)

(21) Appl. No.: 18/321,651

(22) Filed: May 22, 2023

Related U.S. Application Data
(63) Continuation of application No. 12/770,436, filed on Apr. 29, 2010, now Pat. No. 12,014,328.
Application No. 12/770,436 is a continuation-in-part of application No. 11/480,859, filed on Jul. 6, 2006, now abandoned.
Application No. 11/480,859 is a continuation-in-part of application No. 12/352,647, filed on Jan. 13, 2009, now abandoned.
Application No. 12/352,647 is a continuation-in-part of application No. 11/480,859, filed on Jul. 6, 2006, now abandoned.
(60) Provisional application No. 60/698,792, filed on Jul. 13, 2005.
(60) Provisional application No. 61/174,045, filed on Apr. 30, 2009.
(60) Provisional application No. 60/698,792, filed on Jul. 13, 2005.

(51) Int. Cl. F21K 9/235; F21V 23/04

(52) U.S. Cl. CPC G16H 20/13; A61J 7/04; B65D 51/248; G02B 6/0005; G06Q 10/10; G09F 3/208; G09F 7/08; G16H 40/63; G16H 40/67; H05K 5/0017; B65D 2203/10

(57) ABSTRACT

A cap for optical illumination on a container can include a top portion and a base portion having a curved outer surface. The base portion can have a channel formed on at least a part of said curved outer surface. The cap can include a light-emitting diode (LED) electronically connected to circuitry within the cap. The cap can include a light pipe in optical communication with the LED configured to direct light from the LED through the portion of the top portion defined by the light pipe. The cap can include a sensor configured to detect changes in ambient illumination and the LED can turn off when sensor detects the ambient illumination is low.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE DISCLOSURE

This invention relates to medication compliance, and, more particularly, to methods and devices supporting medication compliance.

### INTRODUCTION & BACKGROUND

Medication non-compliance is a major problem in healthcare.

Physicians prescribe medications for a large class of chronic, asymptomatic diseases. These medications must typically be taken daily for the rest of the patient's life in order to sustain quality of life and reduce health risks. Classic examples of diseases in this class include hypertension, hypercholesterolemia and osteoporosis. With many such diseases, a patient feels no different, whether or not they take their medication. So, unlike brushing one's teeth or even exercising, there are no apparent short to medium term costs for noncompliance. This presents a challenge even for those patients who want to comply, let alone those who need a helping hand.

Various attempts have been made in the past to try to increase and improve compliance by patients. Almost all of these systems are essentially reminder systems. For example, there are a large number of pillbox systems that marry alarm clocks to medication containers to remind patients when it is time to take their medication.

While various systems/devices are described here, we do not admit that any of them qualify as prior art to our invention.

There are some compliance intervention systems offered by health care providers designed to remind the patient and alert a remote caregiver. These include a sensor/reminders in the home, a network connection (typically dial-up) to a backend server and outbound messaging/reporting to a caregiver or even back to the patient. These systems, however, are focused on reminding only and while they may include a remote non-professional caregiver in the reminder loop, forgetfulness is only part of the problem.

Other systems try to help patients manage complex medicine regimens. For example, the MD2 device by Interactive Medical Developments of Aurora Healthcare is a coffee maker sized device that stores and dispenses pills like a common gumball machine. The MD2 offers prerecorded audio messages to the patient and network connectivity back to a monitoring service. The MD2 is not designed to be portable, to be wirelessly connected to a network, to relay visual queues to another device resident in the home, or trigger escalating feedback to the patient. The focus on the MD2 is to arm disease management companies to assist patients on multiple medications and to help them effectively manage their regimen from home.

MedPartner of Honeywell Hommed is a platform that helps patients manage complex medicine regimes. The MedPartner platform accommodates several pill bottles and alerts the patient when pills in their regimen needs be taken. The MedPartner system uses RFID technology to label the bottle and its location in an egg-crate like base station. It is networked to a healthcare provider's monitoring station (say in home care or nursing home environments).

SimPill of South Africa describes a pill bottle employing a GSM transmitter which reports to a cellular network whenever a pill is taken. They advertise that their system includes a “pill bottle which, when opened, delivers an SMS [short message service] text message to a central server. The SMS contains a unique pillbox ID number as well as some information about the battery status of the pillbox. Each SMS is time stamped. The central server receives the incoming SMS and, if it is within the time tolerances set for the pillbox sending the message is simply stored for statistical purposes. Should no message be received within the time tolerances then the server can be set to produce a number of responses (e.g., sending a text message reminder to the patient's handset, sending a text message prompt to a family member or community based care giver, prompting them to visit the patient to ascertain the cause of non-compliance and provide assistance, sending a text message to a clinic based health professional or any other user determined response), or indeed escalate through these responses as time elapses with no incoming message in response to the previous outgoing message. Data on levels of compliance as measured by the device are stored for future analysis and use.” The SimPill device is ultimately another reminder system, based on its developer's theory (expounded on their website), that “[a]n important proportion of non-compliance is caused by the patient simply forgetting to take their medication.” When a patient does not take her medication, SimPill reminds the patient and then, possibly, a caregiver. Like the other reminder/alarm systems, SimPill ignores the more complex nature of non-compliance.

A category of medication compliance platforms has been developed specifically for the clinical trial market. In this market it is critically important to capture the dosing data of patients in order to measure their use and the medications efficacy during a clinical research trial. The price point of these devices is necessarily higher and they are built almost as a medical device to suit the stringent requirements of pharmaceutical manufacturers' clinical research requirements. For example, Informedix of Rockville, Maryland has a suite of products focused on compliance systems for the clinical trial market. Their Med-eMonitor is designed to be a clinical data capture diary and medication dispensing device in one. It has electronically monitored medication compartments and an instructional text screen. The device requires a cradle to upload the data and receive power. In the Med-eMonitor if the patient does not return the device to the base station there is no local or remote escalations to remind the patient to take their medication. The platform does not know if the device is even in the home. This suite of devices is designed for monolithic deployment-pharmaceutical companies deploy them in a research trial with a strict protocol that each subject patient must use to fulfill the requirements of the study.

Aardex (Aardex, Ltd. and associated company Aprex Corporation), a Swiss company offers a smart cap to fit standard vials for clinical trial dose recording. The AARDEX MEMS (Medication Event Monitoring System) product employs inductive and capacitive wireless uploading technologies that require close proximity to a networked base-station in the patient's home to upload to a personal computer or even a remotely networked back-end database. The device includes an LCD (liquid crystal display). In order to upload the data from the monitoring caps, a patient has to place it on back into a specially designed base station.

Some prior systems, e.g., as shown in U.S. Pat. No. 6,771,174, require a local computer system at each patient's home to monitor the patient's drug taking. The computer can contact a pharmacist or emergency services if the patient deviates from his or her model behavior. Such systems impose heavy cost requirements—a dedicated computer—at each patient's home. In addition, such systems cannot take advantage of information about other patients, in particular, how other patients have responded to various alert schemes. The inventors were the first to realize that it is desirable and useful to apply techniques to a patient that have been learned from other patients.

U.S. Pat. No. 7,081,807 to Lai discloses an electronic pill reminder device that that is retrofitted inside a regular conventional pill bottle cap—installed inside the conventional pill bottle between the bottle cap and the bottle container. When a user closes the pill bottle cap on the bottle container, an electronic timer, with a factory predetermined time interval, is activated. The timer generates alert signals to remind a user that a last pill has been taken and to remind the user to take his next dose.

The present invention improves on prior systems and overcomes their deficiencies.

### BRIEF DESCRIPTION OF THE DRAWINGS

The following description, given with respect to the attached drawings, may be better understood with reference to the non-limiting examples of the drawings, wherein:

FIG. 1A is an overview of a medication compliance system/framework;

FIG. 1B is a logical overview of the medication compliance framework;

FIG. 1C is a logical diagram showing exemplary internal details of a bottle cap with an electronic embedded curved display;

FIGS. 2A-2G show various views of medication containers using a medicine bottle cap with electronic embedded curved display;

FIGS. 3A-3I are various views of an exemplary medicine bottle cap with an electronic embedded curved display;

FIGS. 4A-4E are partially exploded views of the medicine bottle cap with an electronic embedded curved display;

FIGS. 5A-5C are views of a light pipe used in the medicine bottle cap with an electronic embedded curved display;

FIGS. 6A-6C are views of the top portion of the medicine bottle cap with an electronic embedded curved display;

FIGS. 7A-7D are views of the component board of the medicine bottle cap with an electronic embedded curved display;

FIGS. 8A-8C shows the electronic display portion of the medicine bottle cap with an electronic embedded curved display;

FIGS. 9A-9B show the display positioned relative to the board and connector;

FIGS. 10A-10B are views of an interface component of the medicine bottle cap with an electronic embedded curved display;

FIGS. 11A-11B are bottom and side views, respectively, of a gasket used in the medicine bottle cap with an electronic embedded curved display; and

FIG. 12 is a cross-sectional view of the medicine bottle cap with an electronic embedded curved display.

### THE PRESENTLY PREFERRED EXEMPLARY EMBODIMENTS

### Overview

Well-established behavioral medicine research shows that non-compliance with a medication regimen is fundamentally a behavioral psychology problem. The inventors have realized that timely intervention(s) by machine or human may influence the patient and should increase medication adherence rates.

There are several reasons why patients may not comply with their medication regimens. No one reason or set of reasons may apply to all people. People are motivated in different ways and by different things, and it is an unknown and possibly unique mix of factors that will motivate any particular individual to comply. The inventors have realized that any system for creating or supporting medication compliance will preferably be multi-faceted and be able to learn and adapt to each patient during their course of treatment.

Commonly acknowledged reasons for non-compliance include the following:
Lack of doctor-patient accountability.
Medication is too expensive.
Lack of social support.
Patient's complain or perceive difficulty obtaining refills.
Some patients think that they do not need the medication.
Some patients do not know how to use the medication.
Patients forget to take their medication.
Patients complain of unpleasant side effects

The inventors have realized that a solution to the lack of compliance problem should deal with some or all of these factors.

### System Architecture

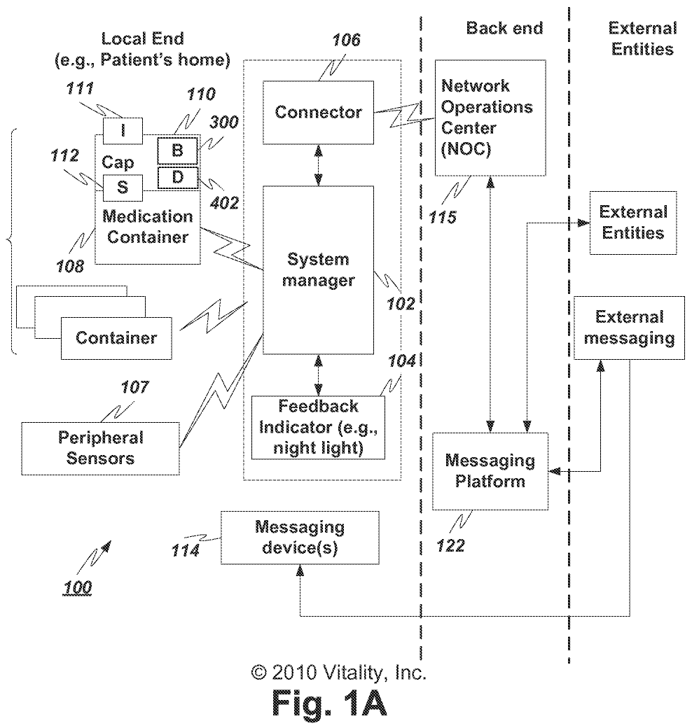

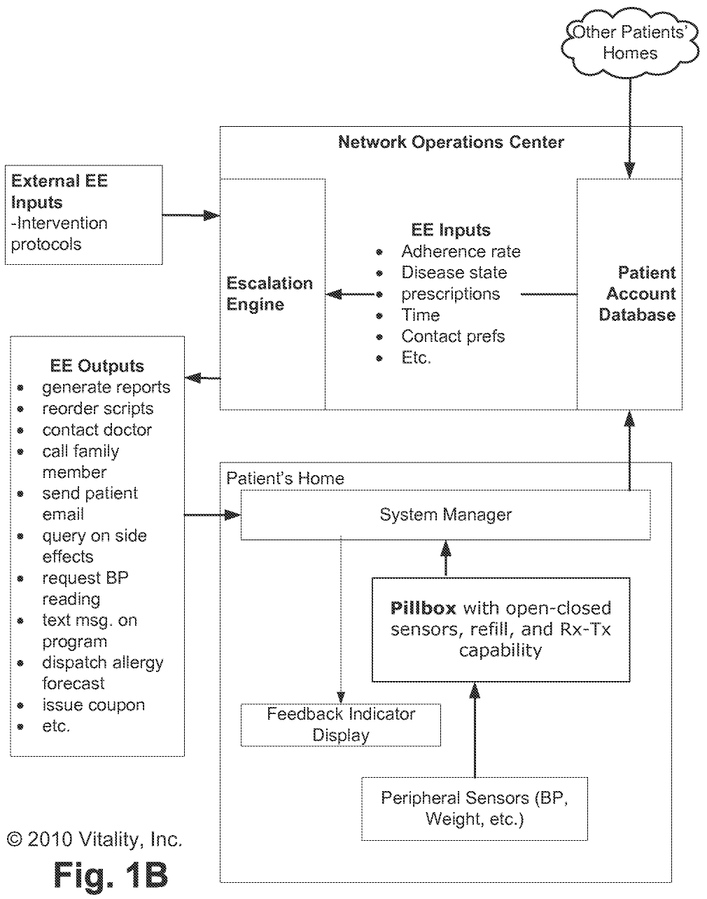

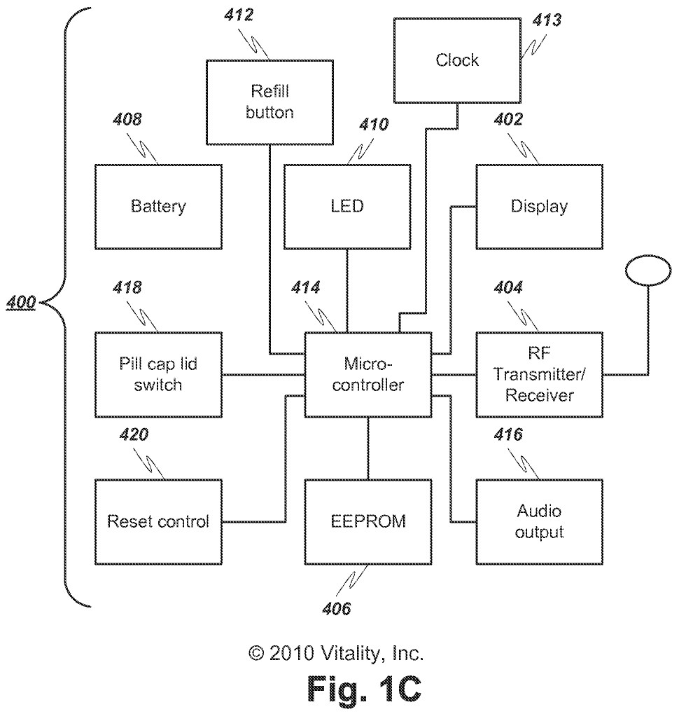

FIG. 1A shows an exemplary medication compliance system/framework 100, and FIG. 1B is a logical overview of the framework. For the purpose of this description, users of the system whose compliance is being monitored and affected are referred to as patients. The use of the word “patient” or “patients” in this description is not meant to limit the scope of the invention or to require any kind of doctor/patient relationship or any other kind of medical or legal relationship with the end users.

A compliance framework can be considered in three logical parts, namely the patients' homes (each a so-called “local end”), a back end, and a part corresponding to external entities that may be involved in the compliance system. The term “patient's home” is used herein to refer to the place (or places) at which a patient is expected to take his medication. It may include, e.g., the patient's home and/or place of work. The patient's home is sometimes referred to herein as the local end.

At a patient's home (or wherever they are supposed to take their medication), the patient is provided with a local system that includes a system manager 102, an optional feedback indicator 104 and a connector 106. The connector 106 allows the local end to connect with the rest of the system (e.g., the back end), and may be a modem, a network connection and the like. Some or all of these components may be integrated into a single device. For example, the system manager 102, a feedback indicator 104, and the connector 106 may be co-located and/or provided in a single device. Alternatively, e.g., the system manager 102 and connector may be formed in a single device. If there is more than one feedback indicator, the system manager may be incorporated into one of them. In a presently preferred embodiment, the system manager 102, feedback indicator 104, and connector 106 are integrated in a single device which includes a “night light” indicator.

The patient's medication is provided in a container 108 with a cap 110. The container 108 may be a regular container or may be specifically adapted to operate with the cap 110. The container/cap combination may be in the form of pill cap, a multi-compartment pillbox, a salve-tube cap, a syringe, an inhaler, a pump dispenser, a drop dispenser and the like. Those of skill in the art will understand, upon reading this description, that the container/cap combination can be used with any medication delivery system and with any type of medication, regardless of its form or dosage. The cap 110 may be fully or partially removable or fully or partially openable, or it may be an integral part of the container through which medication is dispensed. In presently preferred implementations using the cap with the electronic embedded curved display described herein, the container is a regular threaded pill bottle.

At least one sensor 112 is embedded into the medication container 108 and/or the cap 110. The sensor 112 is triggered whenever the container is opened and closed. The sensor may be a pressure sensor, a piezoelectronic sensor, a light sensor, a motion sensor or the like. If more than one sensor is used, the sensors need not all be of the same kind. The function of the sensor(s) is to detect that the medication container has been opened (and then closed). Any sensor(s) (alone or in combination) that achieve this function are acceptable.

Those of skill in the art will realize, upon reading this description, that the container/cap combination may take any form, as long as the system can detect when medication was likely or possibly dispensed.

Although only one medication container 108 is shown in detail in FIG. 1A (for the purposes of this description), it will be understood and appreciated that a patient may have a number of such containers for different medications. Additionally, a particular home (or location) may have medication containers for more than one patient.

A particular system may be open or closed. A closed system will only allow specific and dedicated caps to upload data through the network gateway. (The inventors characterize a closed system as a family of devices in which family members can talk to other family members.) An open system allows all caps to upload their data through the network gateway. (In an open system any member of one family can talk to members of another family.)

Thus, a particular patient may have more than one container (as shown in FIG. 1A), each of which may have a cap and sensors as described above. Those skilled in the art will realize and understand, upon reading this description, that the number and type of containers will depend on the various medications that the particular patient is supposed to take, and that the containers need not all be the same size or type. E.g., some may contain pills; others may contain drops, and so on.

The system assumes that if the medicine container has been opened and then closed, that the medication was actually taken and that the dosage was correct. Preferably the number of pills has to be accounted for upon setup. This known number at the start is decremented by the dosage amount when the cap is opened, and is used to determine whether to initiate a refill.

A local end may also include one or more peripheral sensors 107 to measure and provide data such as the patient's weight, blood pressure (BP), pulse, etc. Peripheral measurements can be provided automatically to the system manager 102 and, in some cases, may be requested by the system manager.

The various containers, sensors and feedback indicators may communicate with the system manager 102 in any known way. The presently preferred implementation uses radio frequencies (ISM band) similar to that used in domestic garage door openers or key fob key-less entry systems. Other protocols such as Bluetooth, ZigBee, Z-wave, 802.11, etc. may be used.

The system manager 102 receives information from and about the sensors in its jurisdiction—the patient's home (in a closed system the system manager will only interact with known sensors). The system manager 102 also communicates with the back end, e.g., via connector 106 using, e.g., a network or a phone system. In some embodiments, the connector 106 is a dedicated telephone dial-up, Ethernet or cellular modem called a network gateway. A network agnostic model may also be used where the network gateway has a plurality of embedded modems and, in the limiting case, the one with the lowest cost of connection, strongest signal or present availability (for instance) defines the connection used. The choice of connection is managed by the central processor or dedicated processor that is in receipt of decision making information from the network(s).

A network gateway is a device that connects the system manager 102 to an external network via POTS (Plain Old Telephone Service) line modem, cellular, pager, 802.11 connections, or the like. In the POTS line modem version, the connector device may be embedded into a so-called “dongle”. In addition to the network connectivity, the dongle may communicate with the system manager over wireless, radio frequency communications.

In a presently preferred embodiment, the connector 106 is a cellular modem that connects to the back end via a cellular telephone network.

The suite of devices described above communicates locally (in the home) and asynchronously from the virtual “backend” system components. Schematically these are local devices that communicate with the backend.

### Backend

The backend is a data service platform that manages individual patients' data. The structure and operation of the backend are described in detail in co-pending U.S. application Ser. No. 12/352,647, filed Jan. 13, 2009, and titled “Medication Dispenser with Automatic Refill,” (Atty. Docket No. 2694-0005), which has been incorporated herein by reference for all purposes.

### Pill Cap

FIG. 1C is a logical diagram showing exemplary internal details of a bottle cap (corresponding to cap 110 in FIG. 1A) with an electronic embedded curved display.

With reference to the diagram in FIG. 1C, in a presently preferred implementation, the pill cap 400 includes an electronic display 402, an RF (radio frequency) transmitter/receiver 404, data store (e.g., EEPROM memory) 406, a power source such as a battery 408, a clock 413, some illumination mechanism (e.g., a tri-color LED) 410, refill button/switch 412, computational resource (computer) 414, and audio output device (e.g., a speaker) 416, a pill cap lid switch 418, a reset control 420, and appropriate circuitry which ties these components together to enable the functional behavior to take place, as described below.

A one-way pill cap only contains an RF transmitter but no receiver. It broadcasts a signal whenever it is opened and then closed within some period of time. Optionally this transmit signal may also be bundled with a payload of data which includes, battery level and a history of last dosing events (e.g., valid close times, where valid is defined to be the time between open and close is short and known) times, unique identification, etc.

A two-way pill cap 400, e.g., as depicted in FIG. 1C (includes a transmitter/receiver 404) transmits a signal whenever it is opened and closed as above. This configuration also enables the cap to receive information from another device downstream which can, e.g., (a) update the cap with new dosing regimen (revised schedule); and/or (b) check if the cap is in range; and/or (c) provided updated information for the display 402. The two-way pill cap is the preferred version, but it requires more software management (overhead) and power. The locally generated information and information received from other devices can be used to update information in the display.

Those skilled in the art will realize, upon reading this description, that different and/or other data may be provided to a one-way cap and to and from a two-way cap.

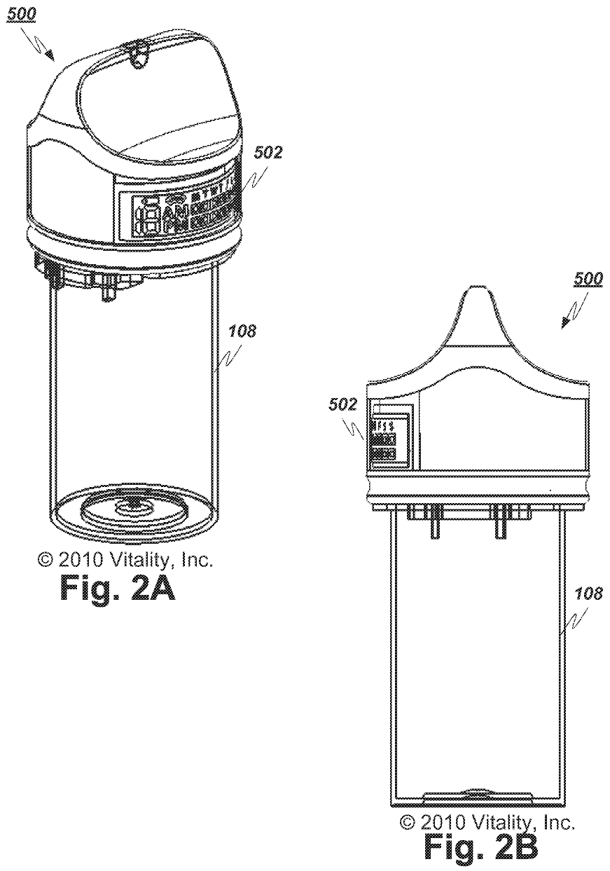

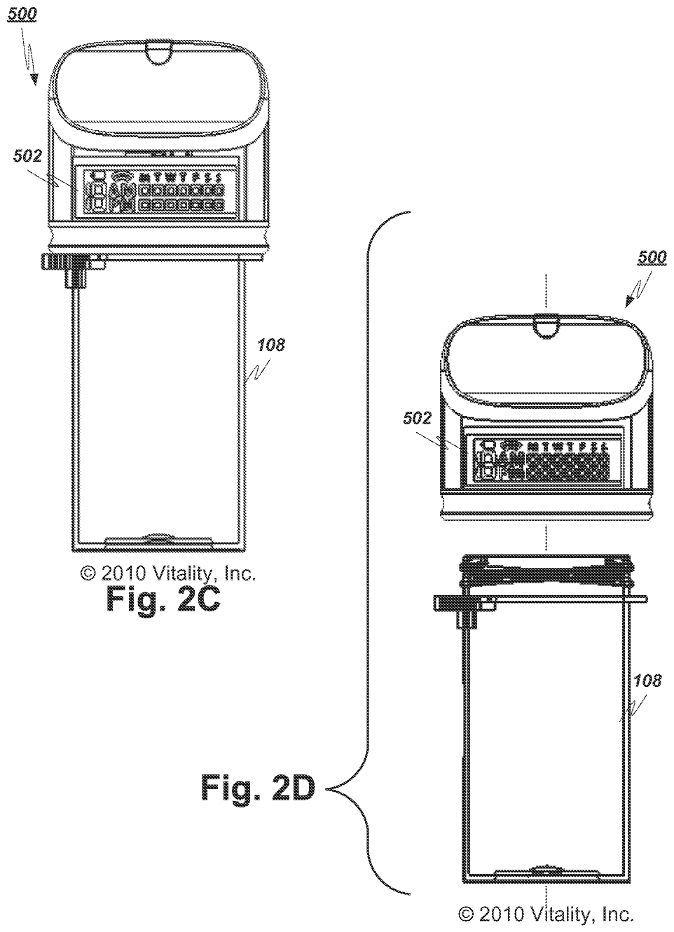

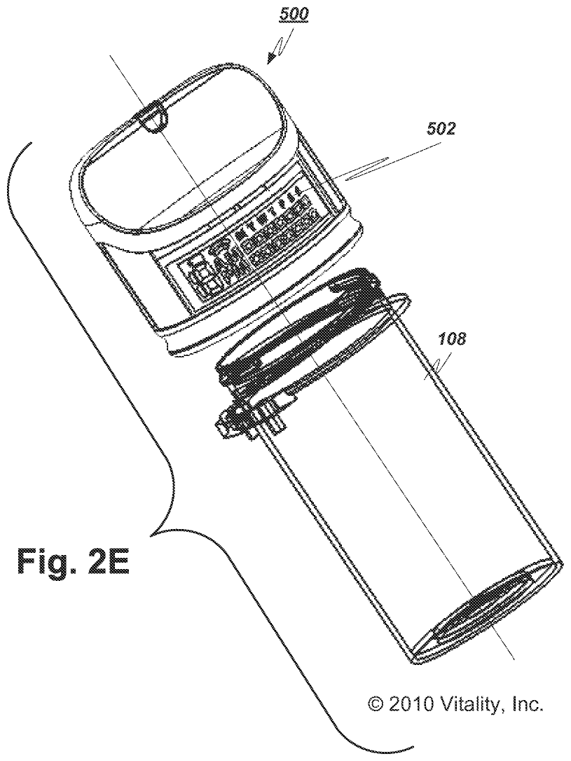

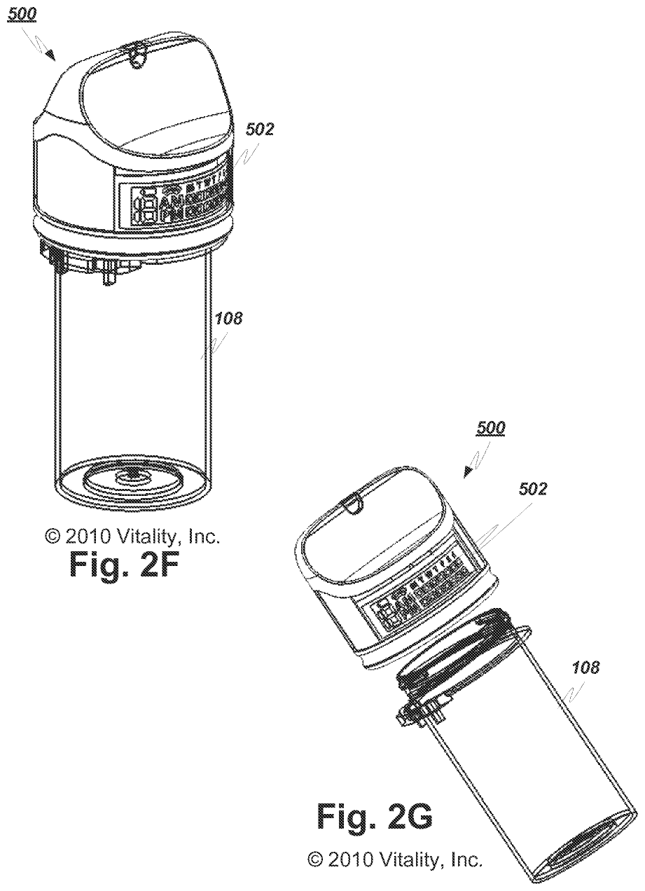

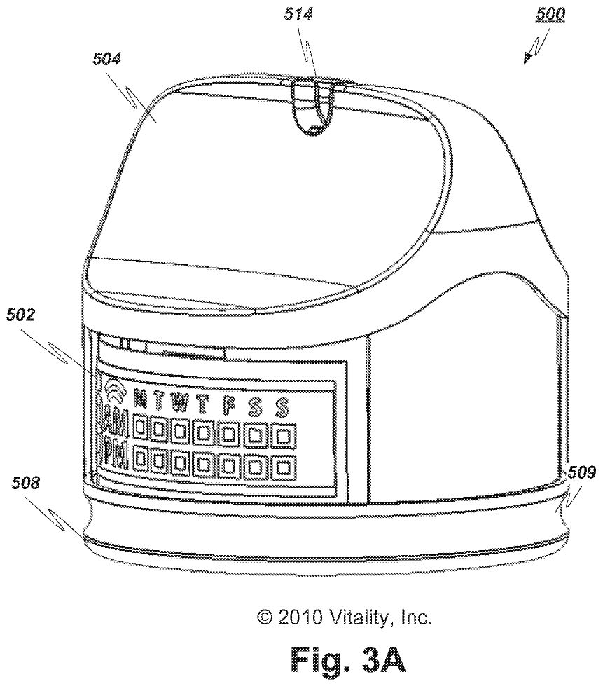

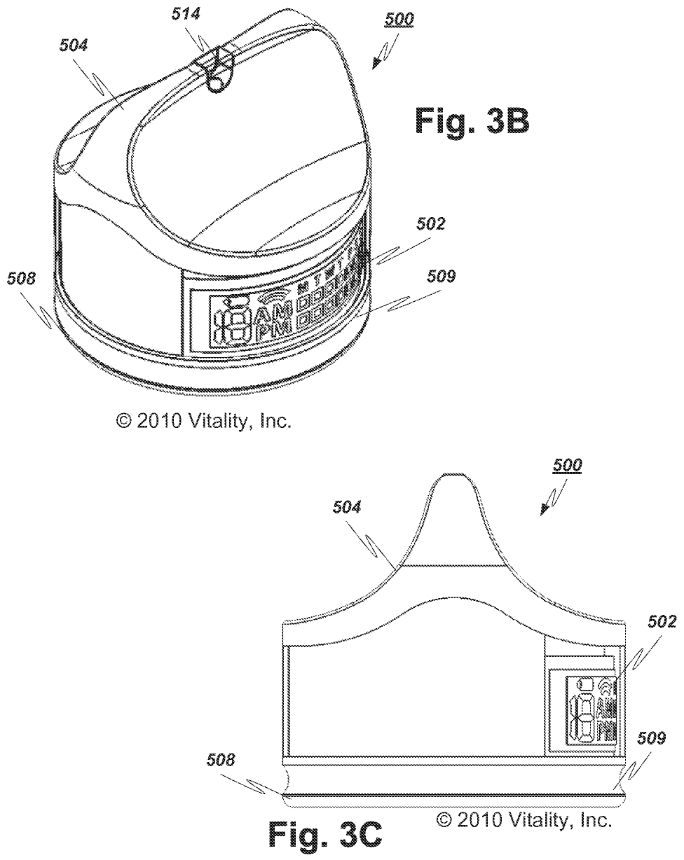

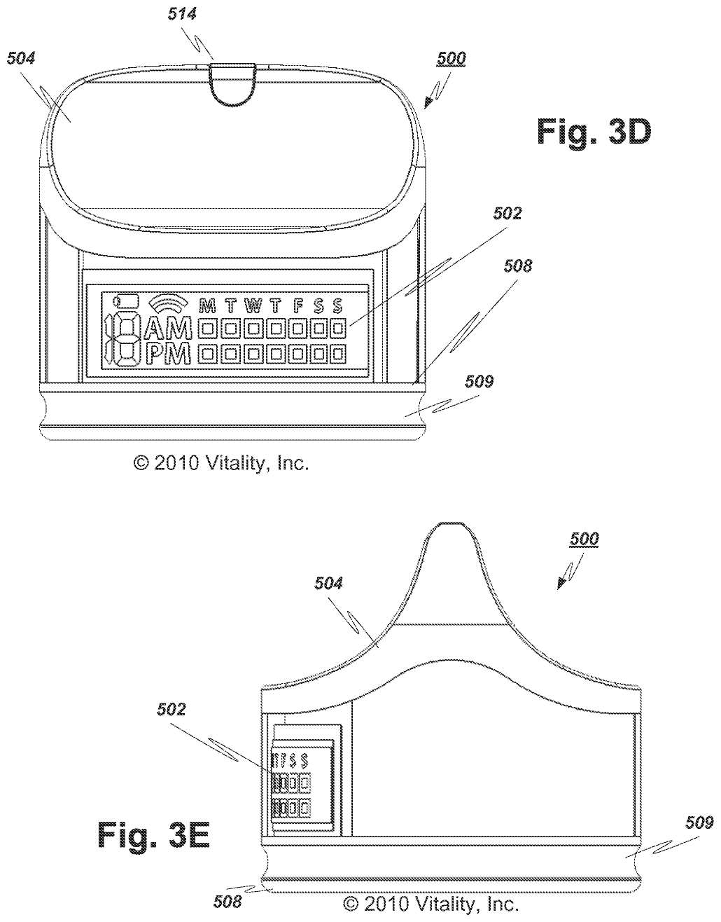

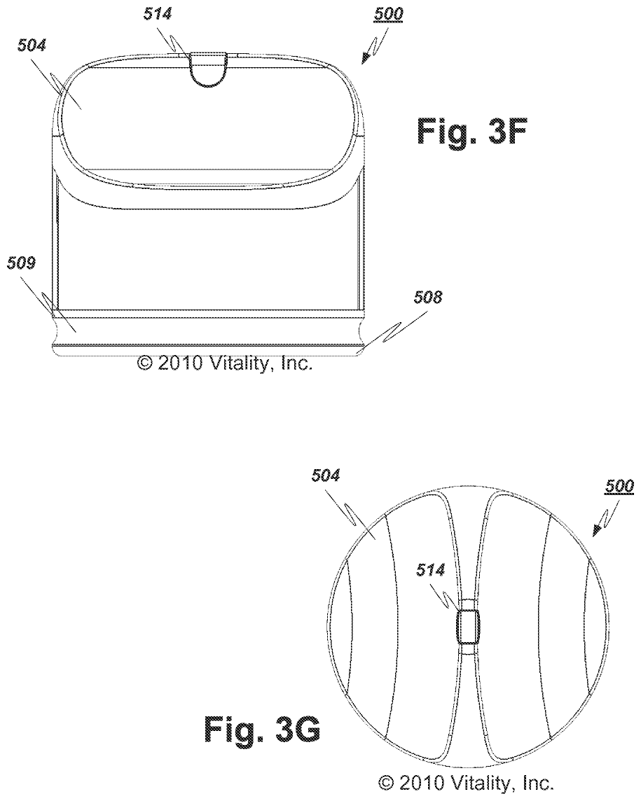

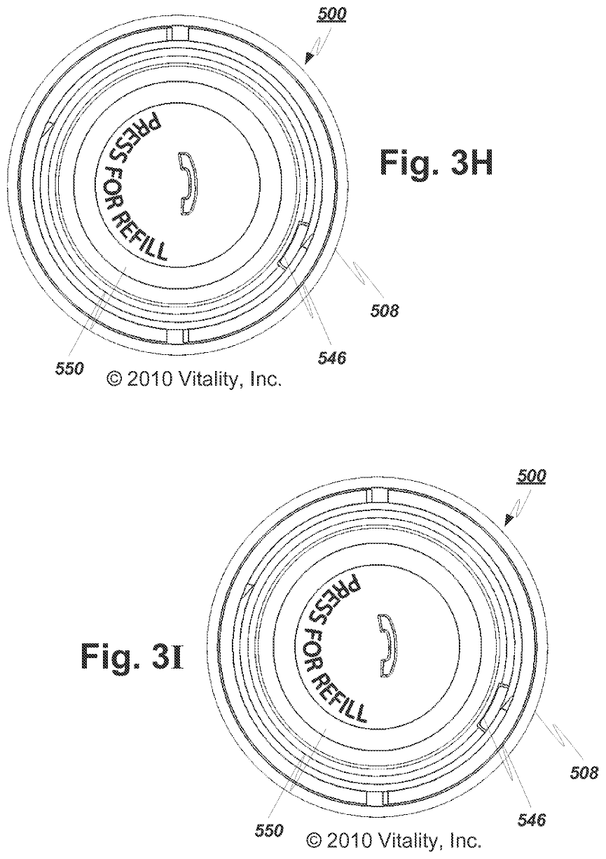

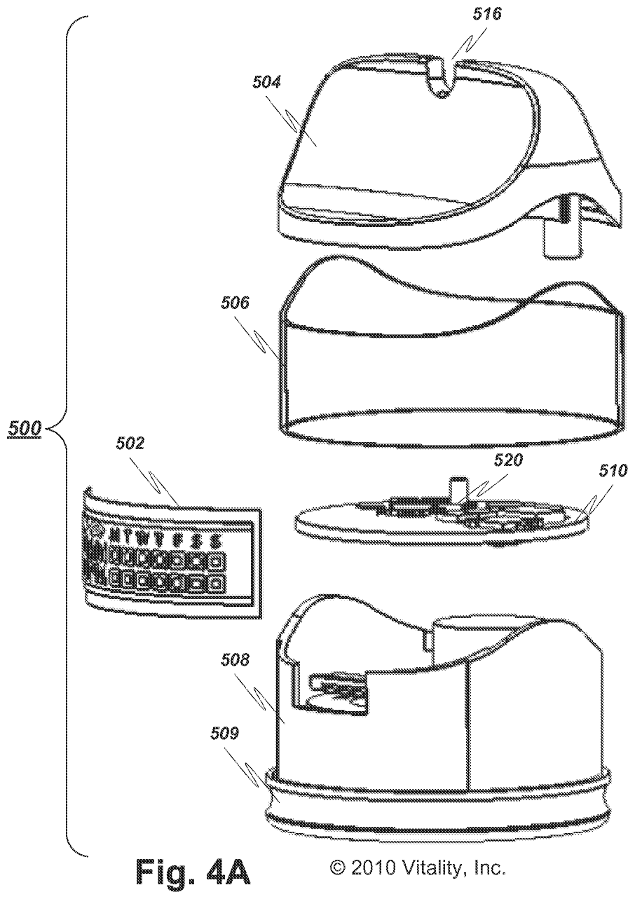

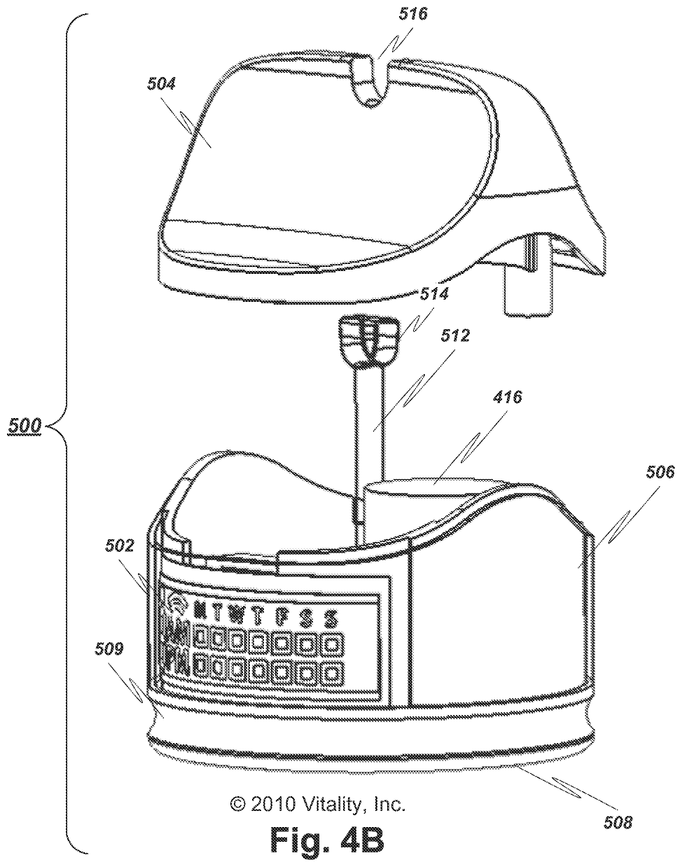

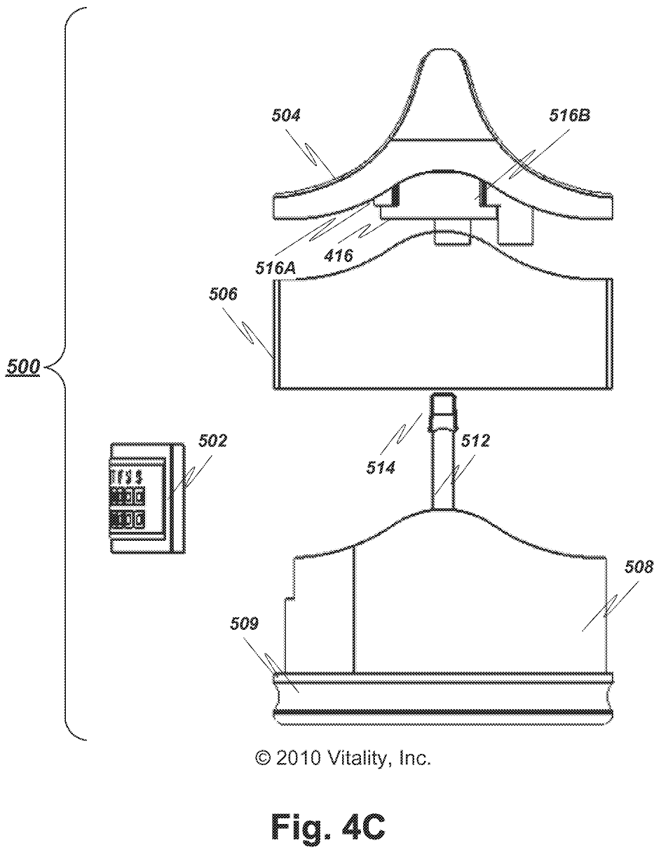

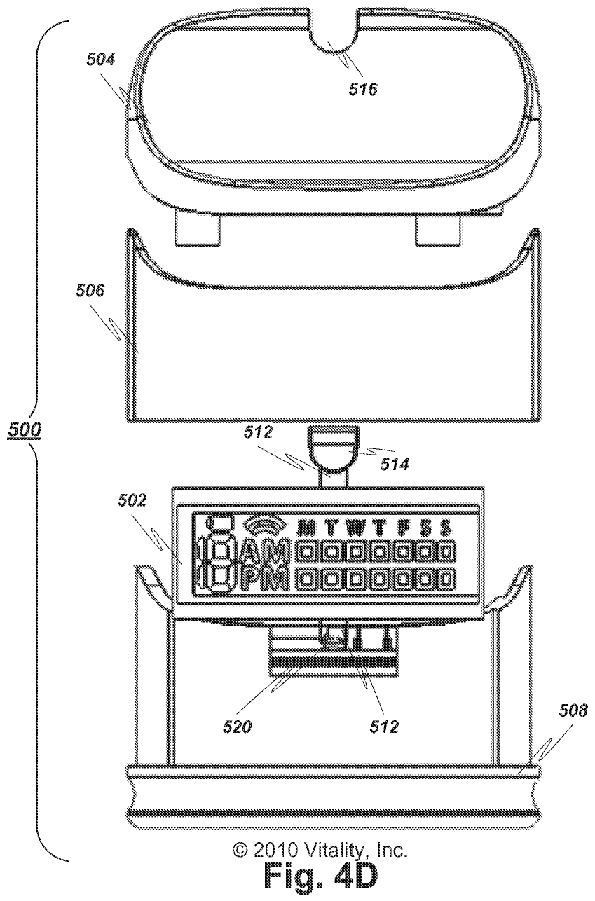

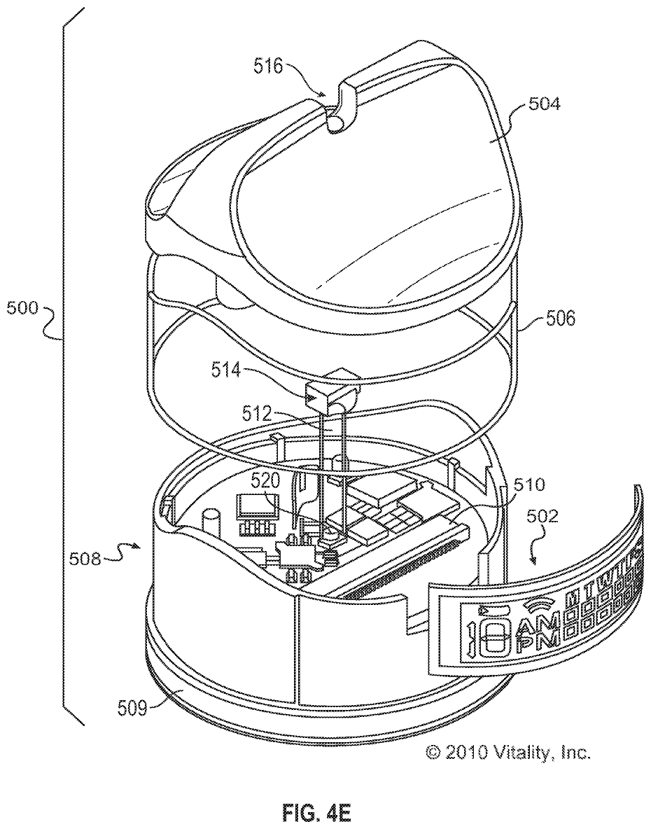

FIGS. 2A-2G show various views of medication containers 108 using a medicine bottle cap 500 (corresponding to cap 400 in FIG. 1C, cap 110 in FIG. 1A) with electronic embedded curved display 502 (corresponding to display 402 in FIGS. 1A, 1C). FIGS. 3A-3I are various views of an exemplary medicine bottle cap 500 with an electronic embedded curved display 502 (shown without the containers 108). FIGS. 4A-4E are partially exploded views of the cap 500 of FIGS. 3A-3E.

With reference to FIG. 4A, the cap 500 includes an electronic curved display 502 (corresponding to display 402 in FIG. 1C), a top cap portion 504, a clear (or optionally tinted) cover 506, and a main body 508. A board 510 and other components (such as some of those shown in FIG. 1C) are contained within the cap. A light-emitting diode (LED) 520 (corresponding to LED 410 in FIG. 1C) is positioned on the board 510, preferably in the center of the board.

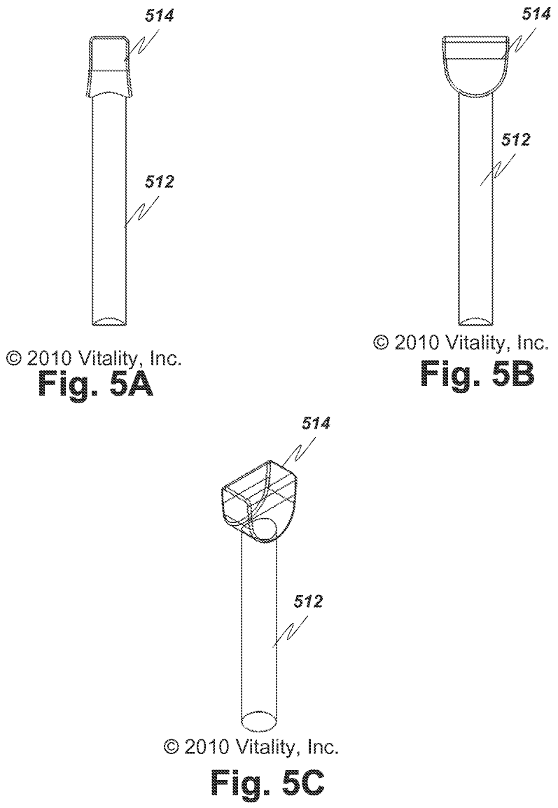

The bottle cap 500 includes a light pipe 512 (not shown in FIG. 4A) having a top portion 514. The light pipe directs light from the light-emitting diode 520 on the board 510 to the outside of the top of the cap. The top cap portion 504 includes a space 516 into which the top portion 514 of the light pipe 512 fits. An exemplary light pipe is shown in FIGS. 5A-5C. Those skilled in the art will know and understand, upon reading this description, that the shape of the light pipe depends on the position of the LED relative to the place on the outside of the cap to which the light is being directed. In the presently preferred implementation, the LED is centered on the board and the light is directed vertically, through the top center of the top portion 504.

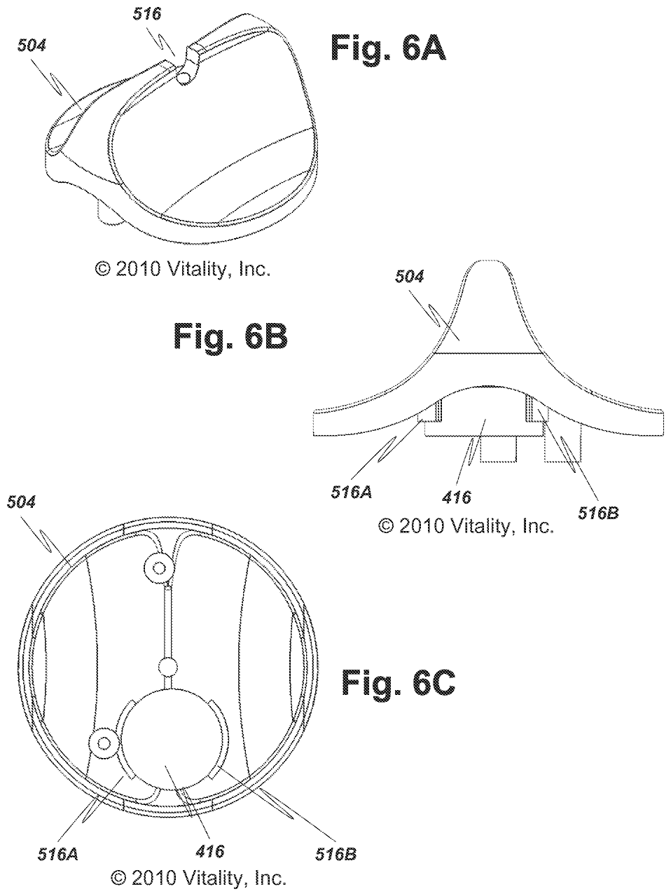

FIGS. 6A-6C are views of the top portion 504 of the cap of FIGS. 3A-3E. The top portion 504 preferably holds the speaker 416. The top portion is preferably formed of hard plastic and includes a mechanism to hold the speaker 416 in place. The speaker in these embodiments is cylindrical (as shown in the drawings), and is held in place by two side-members 516A, 516B formed in the top portion 504. In a presently preferred embodiment the speaker 416 is a cylindrical speaker made by Murata Manufacturing Co., Ltd.

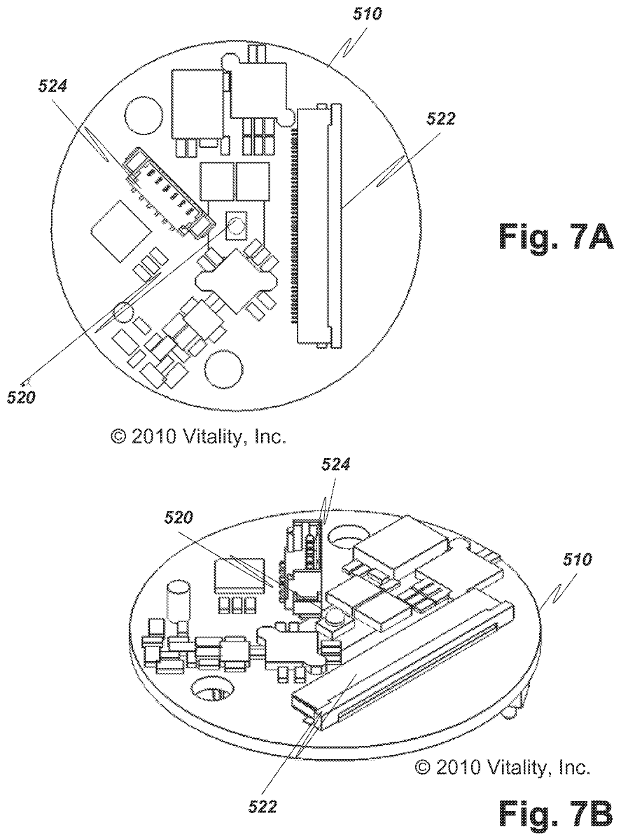

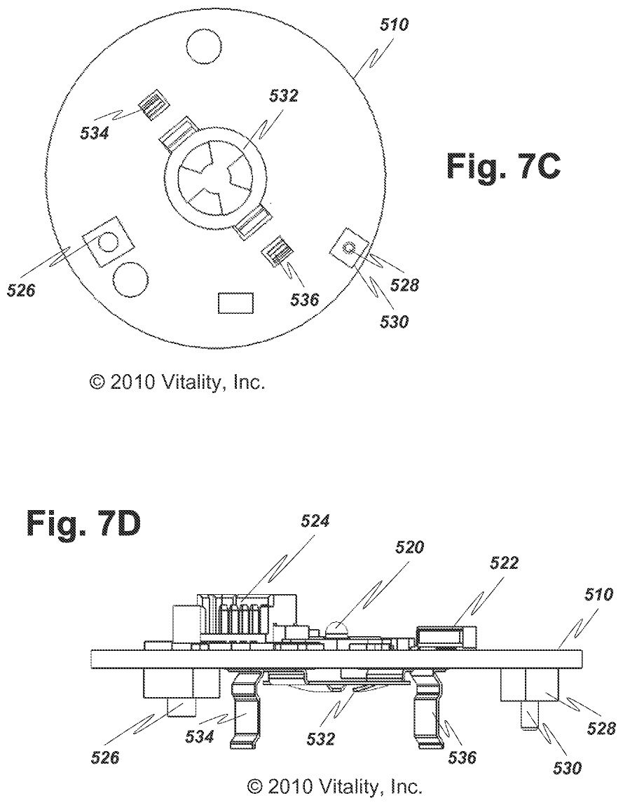

FIGS. 7A-7D are views of the component board 510 of the cap 500. The component board 510 houses various components described above with reference to FIG. 1C, including the LED 520, a connector 522 for the display 502. The board 510 also includes a connecter 524 (Molex programming connector) to allow external connection to the board for programming purposes.

The board 510 also includes RF (radio frequency) transmitter/receiver 404, data store (e.g., EEPROM memory) 406, a clock 413, refill button/switch 412, computational resource (computer) 414, a reset control 420, and appropriate circuitry which ties these components together to enable the functional behavior to take place.

FIGS. 7C and 7D are side and bottom views of the board 510, respectively. The bottom of the board includes switch 526 (corresponding to 418 in FIG. 1C) used to detect removal of the cap from the container. Switch 528 (held inside switch body 530, and corresponding to 412 in FIG. 1C) may be used by a user to manually signal an event/request (e.g., a refill request). The underside of the board 510 also holds a center battery contact 523 and two side battery contacts 534, 536. A battery 408 is help in place against these contacts.

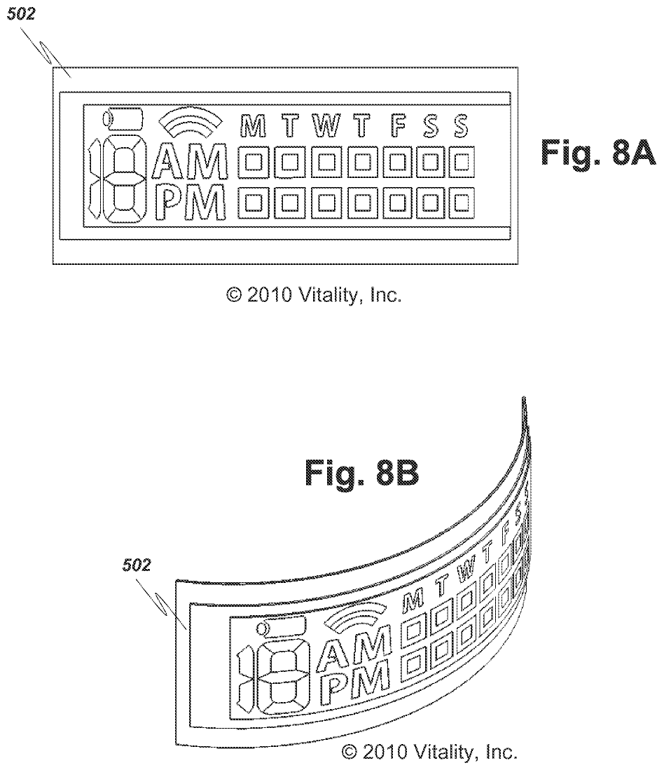

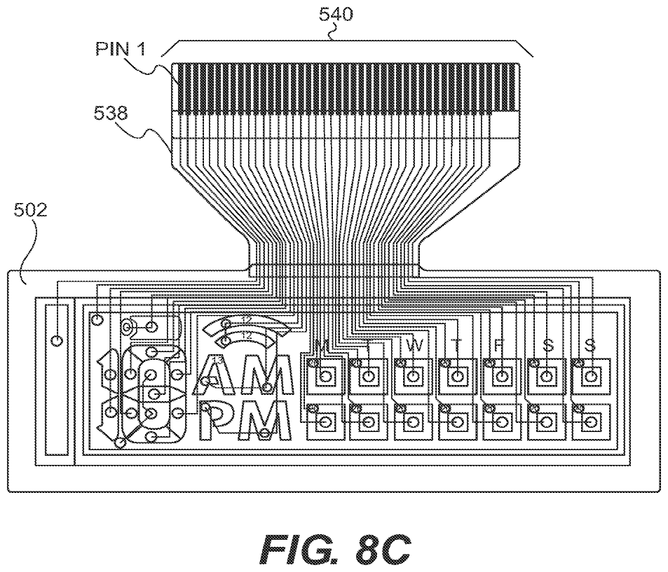

FIGS. 8A-8C show the electronic display portion of the cap of FIGS. 3A-3E. The indicia and glyphs (e.g., day, time, battery, etc.) shown in display 502 (corresponding to display 402 in FIG. 1C) in the drawings are exemplary. Those of skill in the art will know and understand, upon reading this description, that different and/or other indicia and information may be shown on the display 502.

Preferably the display 502 is a flexible display that uses ultra-low power and is easily read under any lighting condition including direct sunlight. Also, preferably the display 502 should look the same from various different viewing angles and should not distort when touched or flexed.

The display is preferably monochrome and uses a fixed set of glyphs, a so-called, segmented display, as is common in the realm of low cost liquid crystal displays. If the number of display segments is very dense, a so called dot-matrix display may be created. In this configuration, the displayed content could become a 2D bar-code readable display enabling it to also function as a machine-readable information display.

In a presently preferred implementation the display 502 is made by E Ink Corporation of Cambridge, Mass. The display 502 is flexible and so curves with the curve of the main body 508. In presently preferred embodiments, the display is about 0.375±0.020 μm thick at its thickest part.

The E Ink display features a membrane of sandwiched charged microspheres that may be rotated to indicate information by changing the charge in a fluid that surrounds the microspheres. Properties of this display that make it desirable for this invention are its extreme low power requirements, mechanical stability, wide viewing angle and sunlight readability. In addition, this invention benefits from this display technology being ultra thin, lightweight and flexible.

To control the display an array of driver wires (538 in FIG. 8C) are connected from the processor (via connector 522) to the display's backplane. In a presently preferred embodiment, this ribbon of driver wires penetrates the exterior wall of the cap to connect to the processor and related circuitry inside (via connector 522).

To maintain the curvature of the cap, the display preferably fits into a receiver channel in the base 508. To make the display appear as if it is on the exterior surface of the cap and not protruding from that surface, a shallow bezel may receive the display so that its top surface shares a common radius with the cylindrical caps exterior. Accordingly, an appropriate indent is preferably formed in the main body 508 to hold the display in place against the body.

To protect the display from damage due to handling, etc., a optionally transparent, wrapper (clear portion 506) covers the display. This wrapper may be selectively tinted as a pneumonic device for the cap owner to recognize which medicine is in the container using the cap. E.g., the transparent wrapper may be tinted a color (e.g., such as red) to help the user identify the medication. Accordingly, the clear portion 506 fits over the display and main body and keeps the display in place and protects it.

The presently preferred connector 522 is a 45-contact connector made by FCI (part number 62684-451100ALF).

While the display is shown in the drawings as going around only a part of the main body (and thus the cap), those of skill in the art will appreciate and understand, upon reading this description, that the display can have different dimensions in length and height, and that the display can cover a greater or smaller portion of the cap. In addition, while the cap is shown with only one display, those of skill in the art will appreciate and understand, upon reading this description, that more than one display can be used, though each display could require its own connector on the board 510.

FIG. 8C shows the connector wiring 538 used to connect the display 502 to the connector 522 on the board 510, and the following table (Table 1) summarizes the wiring of the pins 540 with respect to the connector 522 in a current implementation. (The pins 540 in FIG. SB are numbered 1-45, from left to right.)

TABLE 1

| Pin | Segment description |
|---|---|
| 1 | top plane |
| 2 | Field |
| 3 | One |
| 4 | Bottom left |
| 5 | Battery |
| 6 | Top left |
| 7 | Top |
| 8 | Middle |
| 9 | Bottom |
| 10 | Top right |
| 11 | Bottom right |
| 12 | Antenna signal |
| 13 | AM |
| 14 | PM |
| 15 | “M” bottom middle |
| 16 | “M” bottom outside |
| 17 | “M” top outside |
| 18 | “M” top middle |
| 19 | “T” bottom middle |
| 20 | “T” bottom outside |
| 21 | “T” top outside |
| 22 | “T” top middle |
| 23 | “W” bottom middle |
| 24 | “W” bottom outside |
| 25 | “W” top outside |
| 26 | “W” top middle |
| 27 | “T” Bottom middle |
| 28 | “T” Bottom outside |
| 29 | “T” top outside |
| 30 | “T” top middle |
| 31 | “F” bottom middle |
| 32 | “F” bottom outside |
| 33 | “F” top outside |
| 34 | “F” top middle |
| 35 | “S” bottom middle |
| 36 | “S” bottom outside |
| 37 | “S” top outside |
| 38 | “S” top middle |
| 39 | “S” Bottom middle |
| 40 | “S” bottom outside |
| 41 | “S” top outside |
| 42 | “S” top middle |

Pins 43, 44, 45 have no connection

The mapping of the pins shown in Table 1 above is implementation dependent. Thus, while a specific wiring to the indicia/components in the display is shown in the drawing, those of skill in the art will know and understand, upon reading this description, that different and/or other wiring may be used, depending on the display.

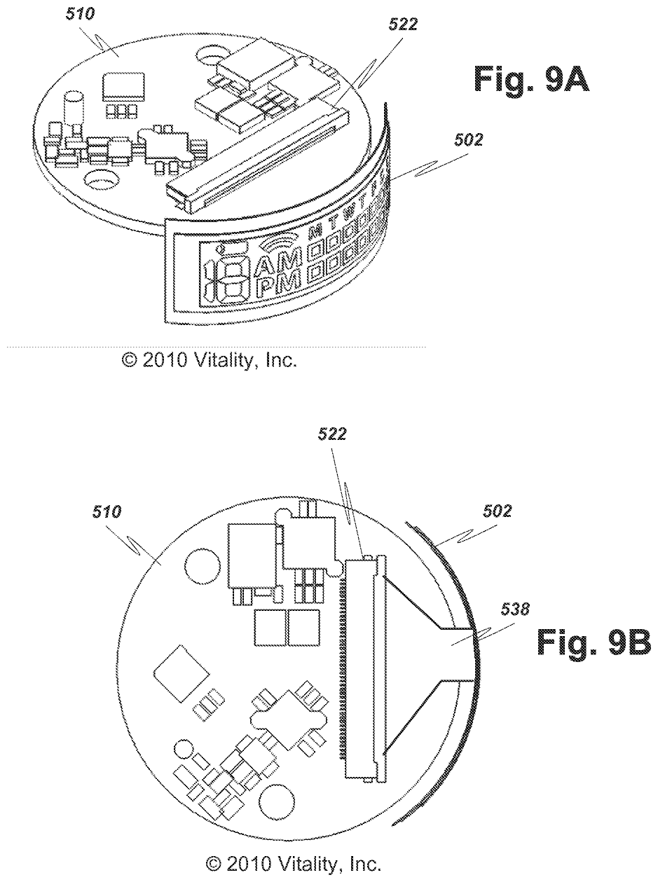

FIGS. 9A-9B show the display 502 positioned relative to the board 510 and connector 522 with wires 538 electronically connecting the display to the connector.

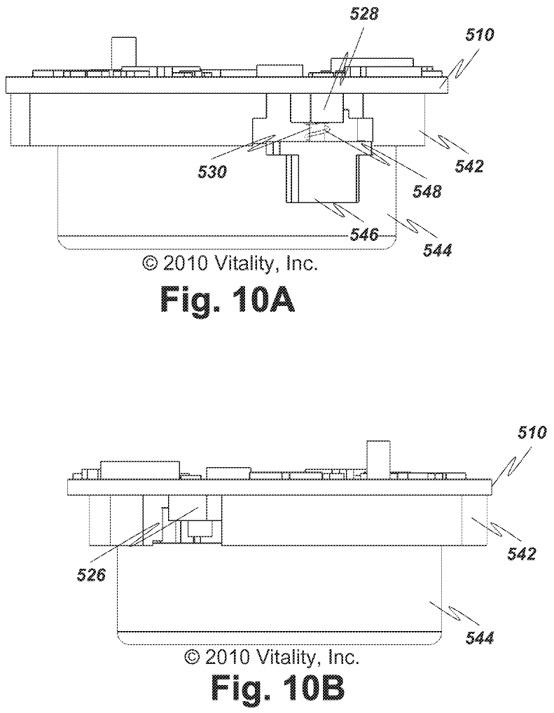

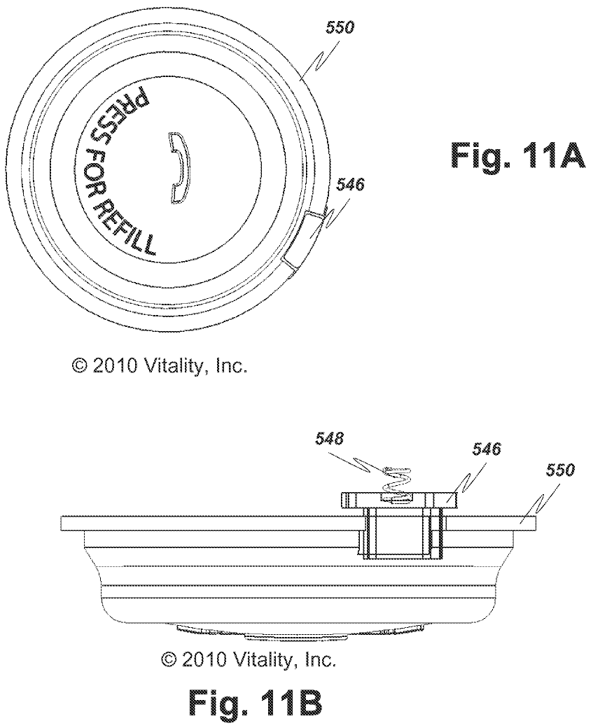

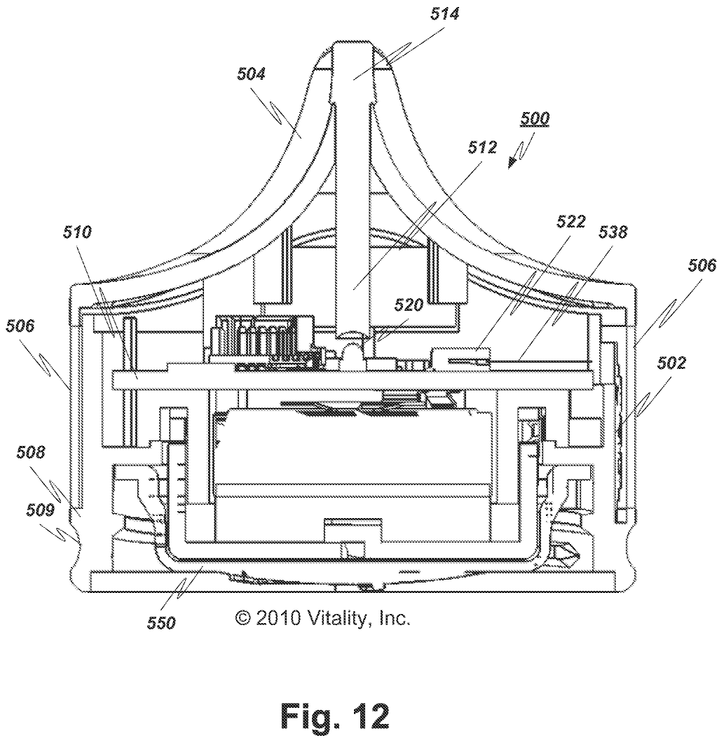

FIGS. 10A-10B are views of an interface component 542 of the cap 500 of FIGS. 3A-3E. The interface component 542 is positioned under the board 510 to position the battery (not show) against the underside of the board and to hold various components. A battery cover 544 holds the battery in place.

Switch 528 (corresponding to 412 in FIG. 1C) held inside switch body 530) may be used by a user to manually signal an event/request (e.g., a refill request). A spring 548 keeps button 546 from depressing the switch 528 without appropriate pressure. A gasket 550 covers the batter (not shown in FIG. 10A, shown in FIGS. 11A-11B), engages the button 546 so that when the gasket is depressed by a user, the button 546 depresses the switch 528.

FIGS. 2A-2G show various views of medication containers using a medicine bottle cap with electronic embedded curved display. While a particular connector system (for connecting the caps to bottles) is shown. Those skilled in the art will realize and understand, upon reading this description, that the actual mechanical interlock mechanism (e.g., screw, bayonet mount, snap-on, etc.) used with each cap will depend on the size and kind of medicine bottle as well as the bottle's interlock system. In some embodiments, an adaptor may be provided to allow caps for one kind of bottle to fit on another kind of bottle.

The main body main include an indented circular portion 509. This portion may be color coded to provide additional information to the patient.

The patient's medication is provided in a container with a cap. The container may be a regular container or may be specifically adapted to operate with the cap. Those of skill in the art will understand, upon reading this description, that the container/cap combination can be used with any medication delivery system and with any type of medication, regardless of its form or dosage. The cap may be fully or partially removable or fully or partially openable, or it may be an integral part of the container through which medication is dispensed.

Those of skill in the art will realize, upon reading this description, that the container/cap combination may take any form, as long as the system can detect when medication was likely or possibly dispensed.

In a preferred embodiment the pill cap includes a light sensor that can detect changes in ambient illumination. This is part of a further battery saving scheme that enables the illuminator to turn off if the container is stored in a dark place. Patients often store their medication in a closed cabinet or drawer (much medication should be stored in a dark place) and there is no reason to deplete battery illuminating the feedback signal if no one can see it. In this scenario the pill cap immediately gives visual indication that it is dose time (“its me” (as opposed to the other caps for which it is not time to dose now)) if dose time has occurred and the ambient light sensor has indicated a change (suggesting it is in view of patient). The ambient light sensor could be replaced with or supplemented with a motion sensor.

In some embodiments, the sensor also detects that the correct medication dose was actually removed from the container.

The display 502 embedded in the cap can provide textual and other visual feedback to the patient. Data on display 502 may instruct the patient which dose (afternoon/morning, etc.), which pill (shape, color, size) needs to be taken.

The cap includes local data memory and permanent memory. The device preferably includes a “store and forward” architecture to ensure data collected on it has a physical location in which to reside if an upload network connection is not possible for some period of time.

A smart cap version provides interoperable mounting rings or bases to the cap. If needed, coupling rings are provided to enable one type of smart cap to mount to any of a variety of commercially available bottles of near similar opening diameter. This avoids having to develop custom caps for each bottle and enables patients to take this platform and use it for medications provided in vials sold by disparate retail pharmacies.

Thus is described a bottle cap having an embedded electronic display that curves around the cap's exterior. The cap is operable in service of promoting medication adherence with a novel feedback loop, e.g., as described in the related patent applications which have been incorporated herein by reference. A cap preferably includes at least the following functional components:
battery,
processor,
open/close switch,
some mechanism for keeping time; and
the curved electronic display.

The display may provide useful information, e.g., an indication of whether the cap has been opened and may optionally indicate a schedule of past open events or indicate future events. The display may also display other information such as, e.g., instructions, medicine name, battery level, time, network connectivity strength (if RF connectivity is also embedded in the cap), patient name, availability of rewards, financial incentives, social network status, who the data is shared with or other such medically relevant information.

In a preferred embodiment, the cap also has network connectivity via standard means (local RF or intermittently via physical connector). In this configuration, among other messages, e.g., the display may indicate environmental conditions such as pollen forecast for allergy related medication, biometrics such as blood pressure for hypertension patients or whether a refill is pending pickup or has been ordered.

As used herein, the term “medication” refers to any kind of medicine, prescription or otherwise. Further, the term “medication” includes medicine in any form, including, without limitation pills, salves, creams, powders, ointments, capsules, injectable medications, drops, vitamins and suppositories. The scope of this invention is not limited by the type, form or dosage of the medication.

Although aspects of this invention have been described with reference to a particular system, the present invention operates on any computer system and can be implemented in software, hardware or any combination thereof. When implemented fully or partially in software, the invention can reside, permanently or temporarily, on any memory or storage medium, including but not limited to a RAM, a ROM, a disk, an ASIC, a PROM and the like.

While certain configurations of structures have been illustrated for the purposes of presenting the basic structures of the present invention, one of ordinary skill in the art will appreciate that other variations are possible which would still fall within the scope of the appended claims. While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A cap for optical illumination on a container, the cap comprising:
a base portion being connectable to a container;
a top portion positioned above the base portion and coupled to the base portion;
a light-emitting diode (LED) electronically connected to circuitry disposed within the cap; and
a light pipe in optical communication with the LED, a portion of the light pipe forming a part of the top portion and being capable of directing light from the LED through the part of the top portion away from the container.

2. The cap of claim 1 further comprising an RF transmitter disposed within the base portion and electronically coupled with the circuity, wherein the RF transmitter is configured to transmit a signal including data relating to a history of dosing event.

3. The cap of claim 1 further comprising an RF receiver disposed within the base portion and electronically coupled with said circuity, wherein the RF receiver is configured to receive a signal including data relating to one or more of a dosing regimen or display information.

4. The cap of claim 1 further comprising a speaker disposed within the base portion and electronically coupled with the circuitry.

5. The cap of claim 1 further comprising at least one switch electronically coupled with the circuitry.

6. The cap of claim 5 wherein the at least one switch detects removal of the cap from a container.

7. The cap of claim 6 further comprising a button mechanically coupled with the at least one switch and configured to actuate the switch in response to a user depressing the button to manually signal an event via the circuitry.

8. The cap of claim 7 wherein the event comprises a refill request.

9. The cap of claim 1, the cap configured to removably secure to one or more of a bottle, a pill box, a salve tube, a syringe, an inhaler, a pump dispenser, or a drop dispenser.

10. The cap of claim 1 wherein the top portion further includes a pair of opposed surfaces extending toward each other from opposed regions of the base portion, wherein each of the opposed surfaces of the top portion defines an externally concave configuration.

11. The cap of claim 10 wherein the top portion further includes a top surface positioned between the pair of opposed surfaces.

12. The cap of claim 1 wherein the LED comprises a multi-color LED.

13. The cap of claim 1 further comprising an electronic display received in a channel formed on at least a part of a curved outer surface of the base portion, said channel electronically connected to the circuitry disposed within the cap.

14. The cap of claim 13 wherein the electronic display extends around at least part of a perimeter of the base portion.

15. The cap of claim 13, the electronic display comprising:
a flexible electronic e-ink display fitting in the channel of the base portion and forming a curved display surface that follows a curvature of the base portion, wherein the e-ink display comprises a membrane of sandwiched charged rotatable microspheres.

16. The cap of claim 13 further comprising a second display distinct from the electronic display, the second display coupled with the circuitry.

17. The cap of claim 13 further comprising a transparent cover covering the electronic display, wherein the transparent cover is tinted with a color.

18. The cap of claim 13 wherein the electronic display is about 0.375 μm thick at its thickest part.

19. The cap of claim 13 wherein the electronic display comprises flexible driver wires configured to couple with a connector of the circuitry.

20. The cap of claim 13 wherein the circuitry is operative to provide visual feedback via the electronic display according to a medication regime.

21. The cap of claim 1 further comprising: at least one optical sensor electronically connected to the circuitry within the cap, the at least one optical sensor configured to detect changes in ambient illumination, wherein the LED is configured to turn off when the at least one optical sensor detects the ambient illumination is low.
